(19) United States
(12) Patent Application Publication
(10) Pub. No.: US 2006/0281777 A1
(43) Pub. Date: Dec. 14, 2006

(54) Process for preparing crystalline form I of cabergoline

(75) Inventors: Sheikh Ahmad, Dearfield, IL (US); Tomasi Attilio, Milano (IT)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/560,877

(22) PCT Filed: Mar. 10, 2003
(86) PCT No.: PCT/EP03/02628

§ 371 Date: Jul. 17, 2006

Related U.S. Application Data
(60) Provisional application No. 60/364,567, filed on Mar. 15, 2002.
(60) Provisional application No. 60/410,163, filed on Sep. 12, 2002.

(51) Int. Cl. C07D 457/02; A61K 31/48

(52) U.S. Cl. 514/288; 546/67

(57) ABSTRACT

A process for producing crystalline form I of cabergoline, which process comprises the preparation of Form V using heptane as precipitation solvent, and its exclusive conversion into crystalline Form I of cabergoline. The present crystallization process from toluene-heptane solvent system for form V involves “reverse addition” of toluene-cabergoline concentrate to cold heptane.

### BACKGROUND OF THE INVENTION

Cabergoline is an ergoline derivative interacting with D₂ dopamine receptors and is endowed with different useful pharmaceutical activities and it is used in the treatment of hyper-prolactinemia, central nervous system disorders (CNS) and other related diseases.

Cabergoline is the generic name of 1((6-allylergolin-8β-yl)-carbonyl)-1-(3-dimethylaminopropyl)-3-ethylurea, described and claimed in U.S. Pat. No. 4,526,892. The synthesis of cabergoline molecule is reported also in Eur. J. Med. Chem., 24, 421, (1989) and in GB-2,103,603-B.

Cabergoline Form I, like cabergoline, displays a significant inhibitory effect with regard prolactine and has therapeutic properties that make it possible to treat patients who have pathological conditions associated with an abnormal prolactine level, thus is useful in human and/or veterinary medicine. Cabergoline is also active, alone or in combination, in the treatment of reversible obstructive airways diseases, for controlling intra-ocular pressure and for the treatment of glaucoma. It is also employed in the veterinary field, as antiprolactin agent and in cutting down drastically the proliferation of vertebrate animals. The several uses of cabergoline are for example described in WO99/48484, WO99/36095, U.S. Pat. No. 5,705,510, WO95/05176, EP040,325. Cabergoline Form I is particularly useful in the treatment of Parkinson's disease (PD), Restless Legs Syndrome (RLS), treatment of diseases like Progressive Supranuclear Palsy (PSP) and Multysystemic atrophy (MSA).

Crystalline cabergoline Form I, an anhydrous not solvated form of cabergoline, was firstly prepared by crystallization from diethyl ether, as described in Il Farmaco, 50 (3), 175-178 (1995).

Another process for preparing crystalline Form I of cabergoline through a toluene solvate Form V was described in WO01/70740. The yield from this process is typically about 60%. For purposes of lowering the cost of the bulk, it is highly desirable to improve the yield of the industrial production of crystalline Form I of cabergoline and to more easily control the de-solvation profile for form V during large-scale manufacturing. Therefore, it is an object of the present invention to obtain a highly pure Form I of cabergoline using an organic solvent system that has never been heretofore used. Efficiently preparing highly pure cabergoline in crystalline Form I in yields exceeding 90% provides benefits with respect to industrial costs and environmental considerations. Moreover, a distinct, unique and desirable de-solvation behavior for the resulting form V towards the isolation of form I was discovered.

### SUMMARY OF THE INVENTION

The present invention concerns a new process for preparing crystalline Form I of cabergoline.

The method of the present invention comprises the preparation of Form V using heptane as precipitation solvent, and its exclusive conversion into crystalline Form I of cabergoline. The present crystallization process from toluene-heptane solvent system for form V involves “reverse addition” of toluene-cabergoline concentrate to cold heptane.

In a second aspect, the invention provides a new process for preparing solvated pure crystalline Form V of cabergoline through phase conversion of initial amorphous precipitate into form V under kinetic control and, in a third aspect, a process for preparing pure crystalline Form I of cabergoline from solvated crystalline Form V of cabergoline based on the use of heptane as suitable solvent for washing the form V prior to de-solvation in the oven.

### BRIEF DESCRIPTION OF THE DRAWINGS

FIG. 1 is an x-ray powder diffraction (XRD) pattern showing peaks characteristic of crystalline cabergoline solvate Form V, made in accordance with Example 1.

FIG. 2 is an x-ray powder diffraction (XRD) pattern showing peaks characteristic of crystalline cabergoline Form I, according to Example 2.

FIG. 3 is a differential scanning calorimeter (DSC) profile of Form V, showing thermal event associated with eutectic melting of cabergoline with toluene.

FIG. 4 is a time resolved x-ray powder diffraction analysis of the de-solvation behaviour of form V made in accordance with example 1, at arbitrarily selected conditions

FIG. 5 is an x-ray diffraction pattern comparison of form I obtained in example 3 with form I obtained in example 2.

### DETAILED DESCRIPTION OF THE INVENTION

According to the present invention, Form I can be readily prepared by a “reverse addition” process starting from crude material. Mechanism for this involves precipitation of amorphous cabergoline followed by phase conversion to form V during the crystallization process. A consequence of this pathway is that form V made through reverse addition has higher free energy than form V made from toluene-di ethyl ether described in the prior art. This results in a distinct de-solvation behaviour for form V made through this new process, which is found to be more conducive for controlled transformation to form I. Use of heptane as wash solvent after filtration, also helps the reduction of toluene content of the wet cake, which in turn facilitates controlled de-solvation of form V to form I in the de-salvation and drying process.

A process for the conversion of form V into crystalline cabergoline Form I is therefore also provided.

The “reverse addition” crystallization procedure could lead to mixtures of form V with amorphous cabergoline, since it involves precipitation of amorphous solids that then phase convert to form V under kinetic control. The amorphous content may not reduce during the de-solvation and drying process. Therefore, there is provided also a method for reducing the amorphous content of either intermediate form V or form I, should mixtures be produced.

The process of the present invention for producing crystalline cabergoline Form I is characterized by crystallisation from a toluene/heptane mixture. Hexane can also be used instead of heptane. Heptane is however, preferred for its toxicological properties, which are better suited for pharmaceutical application.

The process comprises dissolving the raw final cabergoline, obtained as an oil through the synthesis described in Eur. J. Med. Chem., 24, 421, (1989), or any mixture containing crystalline form of cabergoline including Form I crystals obtained from the procedures described in the aforementioned reference, in a suitable amount of a toluene, preferably in an amount of from 2.5 to 4.0 g of toluene per g of cabergoline, more preferably about 3.5 g of toluene per g of cabergoline, at room temperature.

The resulting concentrate is added to cold heptane at temperatures below −10° C., such that there is preferably around 10 to 20 g of heptane per gram of cabergoline. During the addition of cabergoline concentrate, the vessel containing heptane at temperatures below −10° C. is kept under agitation and the intermittent addition rate for cabergoline concentrate to cold heptane is controlled in such a way that all the concentrate is not added in less than 2 hours. With the addition of each droplet of the cabergoline concentrate, solid cabergoline is formed.

However, the initial state of these solids is amorphous in nature, which for the purposes of this invention is defined as a solid form lacking long-range order in three dimensions analogous to crystals. This lack of long-range order is best captured by x-ray powder diffraction analysis. Whilst, x-ray powder diffraction analysis may be best suited to characterize crystalline phases and to detect small amounts of amorphous solids mixed in with crystalline material, polarized light microscopy can also be used to quickly determine if the sample is amorphous or crystalline by those familiar in the art.

The slurry of amorphous cabergoline is stirred at temperatures below −10° C. for no more than three days to phase convert the solids to crystalline form V, preferably for a minimum of 48 hours.

Under these conditions form V is obtained, which may be recovered by common procedures, for example by filtration under reduced pressure or by centrifugal filtration, followed by washing of the solids with pure heptane, preferably 5 mL for each gram of cabergoline, in order to remove residual mother liquor including significant amounts of excess toluene above the molar composition of toluene solvate form V. This facilitates subsequent de-solvation and drying process to make form I.

Form I crystals are obtained by subjecting form V crystals to a de-solvation and drying process for phase conversion and to bring residual toluene at levels acceptable for pharmaceutical use. This can be accomplished by any suitable means such as, but not limited to, heating the solids, reducing the ambient pressure surrounding the solids, or combinations thereof. The drying pressure and time of drying are not narrowly critical. The drying pressure preferably is about 101 kPa or less. As the drying pressure is reduced, however, the temperature at which the drying can be carried out and/or the time of drying likewise is reduced.

Particularly for solids wet with high boiling solvents like toluene, drying under vacuum will permit the use of lower drying temperatures. The optimum combination of pressure and temperature is usually determined from the vapour pressure versus temperature diagram for toluene and operational factors related to the design of the dryer. The time of drying need only to be sufficient to allow for phase conversion of form V to form I and for the reduction in the level of toluene to a pharmaceutically acceptable level. When the solids are heated to remove the solvent, such as in an oven, a temperature that preferably does not exceed about 150° C. is selected.

As stated above, Form V crystals made through the reverse addition process and Form I crystals subsequently obtained after the drying process may contain some amorphous cabergoline. Its level can be reduced to below the typical detection limit of x-ray powder diffraction method by suspending Form V or Form I crystals under moderate agitation, in pure heptane, preferably 20 g of heptane per gram of cabergoline, at a temperature of from 45° to 60° C. for about 4 to 20 hours, preferably for about 24 hours at 45° C. Very small quantities of toluene can also be added to the slurry to further accelerate the conversion of amorphous cabergoline to crystalline cabergoline.

The reduction of the amorphous form content may be also obtained by other “vapour based” methods well known in the art.

The crystals of Form I of cabergoline prepared according to the process of the present invention have preferably a polymorph purity >95%, more preferably >98% at yields in excess of 90% w/w, compared to about 60% for the route described in WO01/70740.

Characterisation

X-ray powder diffraction (XRD) was used to characterise the solvate Form V and form I of cabergoline.

X-Ray Diffraction Analysis

Powder X-ray diffraction was performed using either a Siemens D5000 powder diffractometer or an Inel multi-purpose diffractometer. For the Siemens D5000 powder diffractometer, the raw data were measured for 2θ (two theta) values from 2 to 50, with steps of 0.020 and step periods of two seconds. For the Inel multi-purpose diffractometer, samples were placed in an aluminium sample holder and raw data were collected for one thousand seconds at all 20 values simultaneously.

It is worth mentioning that while peak positions in x-ray powder diffraction reflect the three-dimensional long order within a crystalline form defined by its lattice parameters and must be the same for a given solid form, relative peak intensities do not solely reflect the internal order or structure. Relative intensities can be affected by attributes such as differences in the external shape of the crystals of the same form, which in turn can be altered by process conditions pertinent to the crystallization of a given form. Furthermore, sample preparation prior to x-ray diffraction analysis can also lead to differences in the relative intensities for the same solid form.

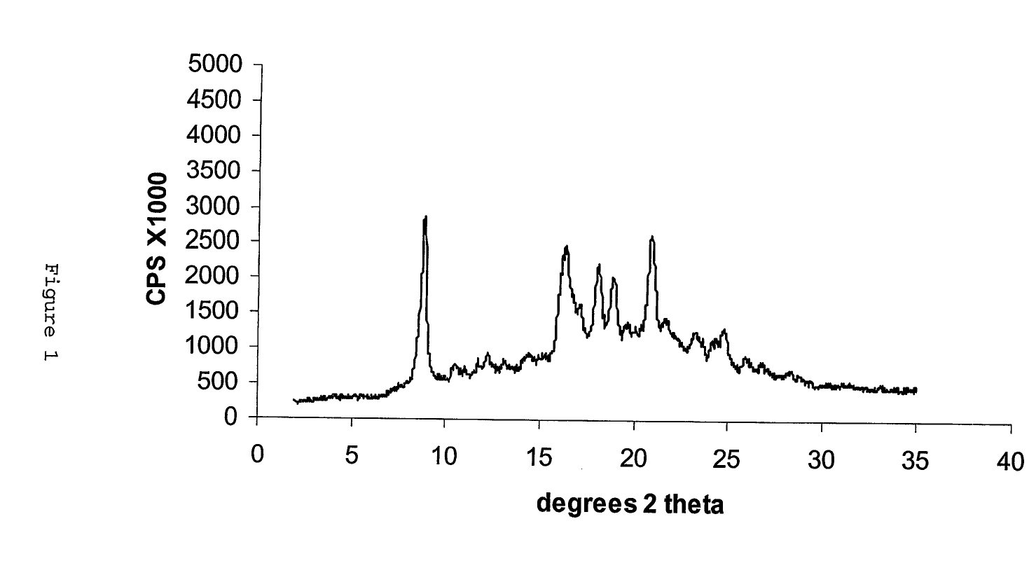

The x-ray powder diffraction pattern for cabergoline Form I (FIG. 1) made according to example 1 and obtained from the Inel multi-purpose diffractometer shows a crystalline structure with distinctive peaks depicted in the following table I. Percent peak intensities in table I are calculated after correcting for the hump (reflective of some amorphous cabergoline mixed in with form I) in the baseline of x-ray powder diffraction pattern of form I shown in FIG. 1.

TABLE I
X-Ray diffraction data, Form I

| Angle | Intensity | |
|---|---|---|
| 2θ | Cps × 1000 | Intensity % |
| 9.870 | 2394 | 87.86 |
| 10.497 | 577 | 21.17 |
| 12.193 | 537 | 19.70 |
| 14.707 | 849 | 31.17 |
| 16.658 | 756 | 27.74 |
| 16.721 | 788 | 28.91 |
| 18.707 | 2725 | 100.00 |
| 20.822 | 1137 | 41.72 |
| 22.688 | 543 | 19.92 |
| 24.652 | 1407 | 51.63 |

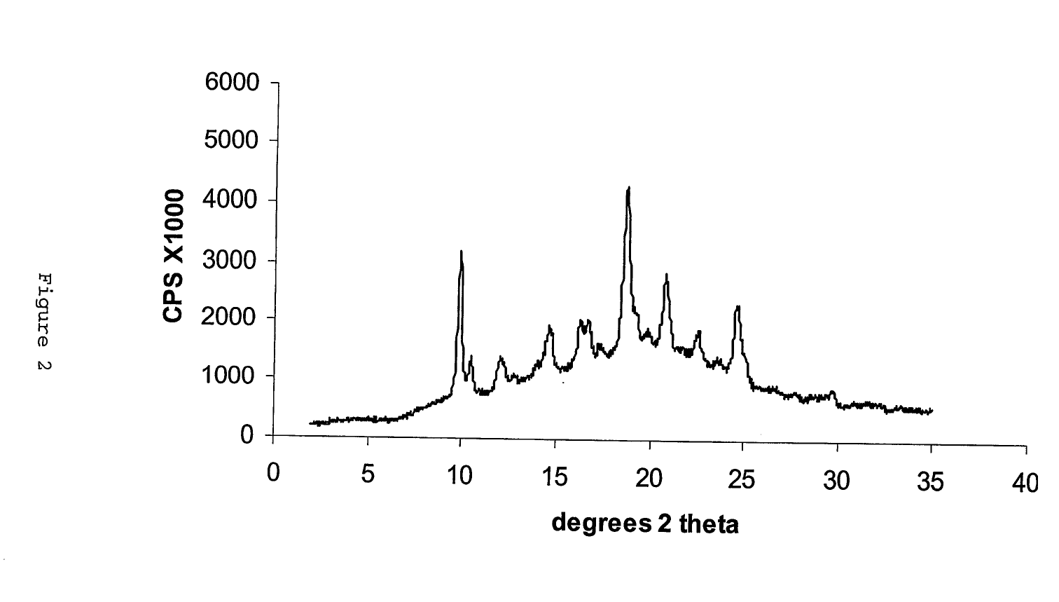

The x-ray powder diffraction pattern for the known toluene solvate Form V of cabergoline made according to example 2 (FIG. 2) and also described in WO01/70740 has a crystalline structure with distinctive peaks depicted in the following table II. Percent peak intensities in table II are calculated after correcting for the hump (reflective of some amorphous cabergoline mixed in with form V) in the baseline of x-ray powder diffraction pattern of form V in FIG. 2.

TABLE II
X-Ray diffraction data, Form V

| Angle | Intensity | |
|---|---|---|
| 2θ | Cps × 1000 | Intensity % |
| 8.866 | 2222 | 100.00 |
| 12.287 | 120 | 5.40 |
| 16.375 | 1242 | 55.90 |
| 18.171 | 887 | 39.89 |
| 18.991 | 700 | 31.50 |
| 21.043 | 1255 | 56.50 |
| 24.938 | 243 | 10.93 |

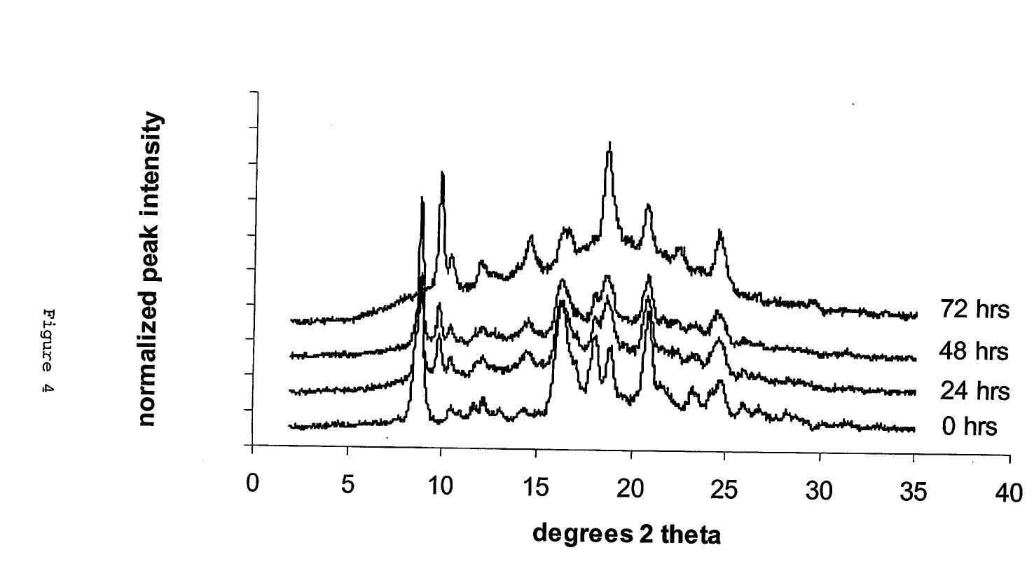

The de-solvation and phase transformation behaviour of Form V prepared in accordance with example 1 to Form I was studied by placing 1.50 g sample of form V in a crystallization dish in a vacuum oven operated at 43° C. and 94.8 kPa vacuum for 48 hours. This drying phase was followed by 24 hours at 57° C. and 94.8 kPa vacuum. Samples were withdrawn every 24 hours for x-ray powder diffraction analysis. FIG. 4 shows the time resolved behaviour under these arbitrarily selected conditions. The data shows, that form V made in accordance with example 1 began converting to form I (characterized by 9.870 and 18.707 degrees 2θ peaks) within 24 hours and the transformation was complete within 72 hours.

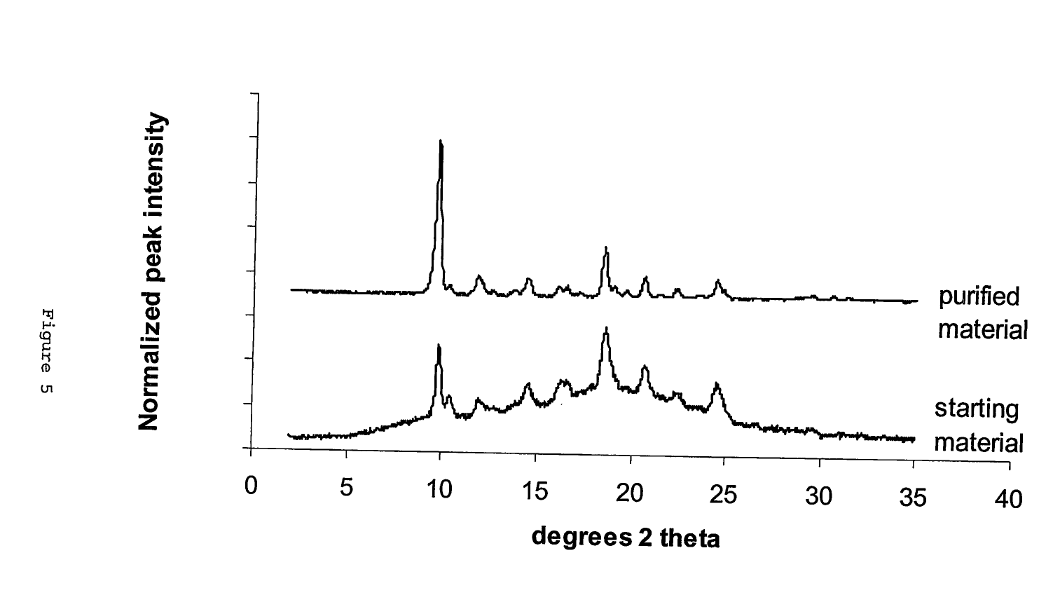

X-ray powder diffraction anlaysis was also used to evaluate the effectiveness of the procedure described in examples 3 for reducing amorphous content of form I that can be obtained through procedures described in examples 1 and 2. FIG. 5 depicts the results of the x-ray diffraction analysis conducted before and after the treatment of form I with the procedure described in examples 3.

Differential Scanning Calorimeter Analysis (DSC)

Differential scanning calorimeter profiles were obtained from a Mettler-Toledo 822^{e} differential scanning calorimeter. The data was collected between 25 and 150° C. at a heating ramp of 10° C./min. Forty micro-liter hermetically sealed aluminium pans with a pinpricked hole in the lid were used.

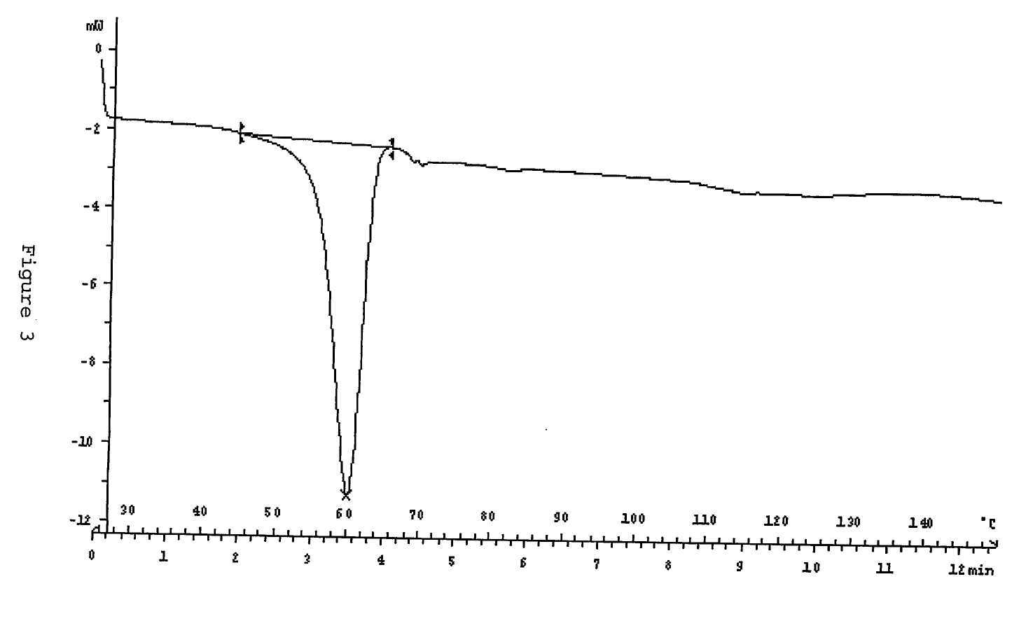

Differential scanning calorimeter profile for Form V (FIG. 3) shows a single endothermic thermal event centred around 62° C. This thermal event corresponds to the eutectic melting of Form V in toluene. For the purposes of this invention eutectic melting is defined as the transformation of solvent containing solids into a homogeneous liquid solution without any significant loss of solvent associated with the solids. Solution calorimetry was performed using a Parr 1455 solution calorimeter to obtain enthalpy of solution data and understand the differences between form V made through the reverse addition process reported here and the procedure for making form V that was described in WO01/70740. The measurements were performed in duplicate at approximately 21° C. by dissolving approximately 0.3 g of form V sample obtained from either process in approximately 100 mL of pure toluene.

Form V made from the reversed addition procedure reported here gave an average value of 23.93 kilo Joules/mole for enthalpy of solution, while form V made by the procedure reported in WO01/70740 gave an average value of 25.56 kilo Joules/mole. The lower values for form V made through the reverse addition procedure indicate that it would exothermically convert to form V crystals obtained through the procedure described in WO01/70740. The reasons for lower enthalpy of solution for form V made through “reverse addition” process would include “reduced molecular order”, possibly resulting from a small amount of amorphous cabergoline mixed in with form V. It is suggested that, the fact that “reverse addition” process crystallizes form V through phase transformation of amorphous cabergoline could lead to small amounts of amorphous cabergoline to persist even after the phase transformation form V is seemingly complete in the slurry. Differences in the enthalpy of solution for form V made through different methods can also have favourable consequences for the de-solvation process that leads to form I.

### EXAMPLES

The following Examples contain detailed descriptions of methods of preparation of crystalline forms of cabergoline described herein. These detailed descriptions fall within the scope of the invention and illustrate the invention without in any way restricting that scope. All percentages are by weight unless otherwise indicated.

### Example 1

### Preparation of Crystalline Form V of Cabergoline

2.0 g of cabergoline were dissolved in 7.01 g of toluene in a 25 mL scintillation vial by agitating with a magnetic bead. In a 125 mL jacketed reactor equipped with an overhead agitation system, cooled 30 g of heptane to a set point of −18° C. in order to achieve a temperature of −15° C. in the reactor. The cabergoline concentrate in toluene was then intermittently added to cold heptane over 2 hours, with the agitation in the reactor set at 203 revolutions per minutes. Agitation was lowered to 175 revolutions per minute upon the completion of the concentrate charge. Solids formed with the addition of every single droplet of the concentrate. These initial solids were confirmed as amorphous by polarized light microscopy. The slurry was stirred for 48 hours at −15° C. after the completion of the cabergoline concentrate charge to phase transform amorphous cabergoline to crystalline form V of cabergoline. After 48 hours the slurry was discharged onto a filtration flask operating under reduced pressure. The cake was washed with 10 mL of heptane to remove mother liquor and wash away excess toluene from the solids. The solids were left on the filter for twenty-five minutes under pressure.

They were identified as form V by XRD, per the data shown in FIG. 1 and table 1. Yield was about 100% (w/w) on the basis of the content of pure “toluene free” cabergoline.

### Example 2

### Preparation of Crystalline Form I of Cabergoline

The toluene solvate form V obtained in example 1 was placed in vacuum oven at 43° C. and under 94.8 kPa of vacuum for 48 hours, followed by 6 hours at 55° C. After drying the overall yield has about 93% on the basis of pure cabergoline initial content and the resultant solid form was identified as form I by XRD. The pattern had all the characteristic peaks listed in table 2, however, it also had a small “hump” in the base line of the x-ray powder diffraction pattern indicative of some amorphous material mixed in with form I (FIG. 2 and the pattern labelled “starting material” in FIG. 5).

### Example 3

### Reduction of Amorphous Content of Crystalline Form I of Cabergoline

To a 12 mL vial equipped with a magnetic bead for agitation, 100 mg of amorphous containing form I obtained in example 2 was added. This was followed by the addition of 2.0 g of heptane. The resulting slurry was stirred for 24 hours on magnetic plate at 45° C. The slurry was then discharged onto a filtration flask operating under pressure. The cake was washed with 1.0 mL of heptane and air-dried for thirty minutes. The solids were analysed by x-ray powder diffraction. They were identified as form I solids, with amorphous cabergoline below the detection limits for x-ray powder diffraction technique (see “purified material” pattern in FIG. 5).

1. A process for producing crystalline Form I of cabergoline, which process comprises the preparation of toluene solvate Form V of cabergoline having the XRD powder pattern of FIG. 2 by “reverse addition” and its conversion into crystalline Form I of cabergoline.

2. A process according to claim 1 in which the reverse addition is the addition of toluene-cabergoline concentrate to cold heptane.

3. A process according to claim 1 in which the the preparation of toluene solvate form V comprises dissolving raw cabergoline, or any mixture containing crystalline form of cabergoline including Form I crystals, in a suitable amount of a toluene at room temperature, adding the resulting concentrate to cold heptane at temperatures below −10° C., keeping under agitation the vessel containing heptane at temperatures below −10° C. and controlling the intermittent addition rate for cabergoline concentrate to cold heptane in such a way that all the concentrate is not added in less than 2 hours, stirring the resulting solution containing solid cabergoline and converting the resulting solvate form V into cabergoline Form I by de-solvation and drying process.

4. A process according to claim 3 in which the suitable amount of toluene is from 2.5 to 4.0 g of toluene per gram of cabergoline.

5. A process according to claim 3 in which the suitable amount of toluene is about 3.5 g of toluene per gram of cabergoline.

6. A process according to claim 2 in which the solution containing solid cabergoline is stirred to a temperature below −10° C. for no more than three days.

7. A process according to claim 2 in which the resultant gel is quenched with cold heptane.

8. A process according to claim 2 in which the final drying is carried out by heating the solids of the solvate form V, reducing the ambient pressure surrounding the solids, or combinations thereof.

9. A process for producing solvate form V of cabergoline having the XRD powder pattern of FIG. 1 which process comprises dissolving raw cabergoline, or any mixture containing crystalline form of cabergoline including Form I crystals, in a suitable amount of a toluene at room temperature, adding the resulting concentrate to cold heptane at temperatures below −10° C., keeping under agitation the vessel containing heptane at temperatures below −10° C. and controlling the intermittent addition rate for cabergoline concentrate to cold heptane in such a way that all the concentrate is not added in less than 2 hours, stirring the resulting solution containing solid cabergoline and collecting the resulting solvate form V of cabergoline.

10. A process for producing crystalline Form I or crystalline Form V without the presence of any detectable amount of amorphous cabergoline, which process comprises suspending Form V or Form I crystals under moderate agitation in pure heptane at a temperature of from 45° to 60° C. for about 4 to 20 hours.

11. A process according to claim 10 characterized in that very small quantities of toluene are also added to the suspension in heptane of Form V or Form I crystals.
